(19) United States
(12) Patent Application Publication
(10) Pub. No.: US 2012/0244529 A1
(43) Pub. Date: Sep. 27, 2012

(54) MULTIPLEX ANALYSIS OF CELLS, PARTICLES, AND OTHER ANALYTES

(76) Inventors: Martin Fuchs, Uxbridge, MA (US); Henrik Stender, Gentofte (DK); Michelle Meltzer, Chelmsford, MA (US); Shepard Janeen, Wilmington, MA (US); Richard Huang, Newton, MA (US)

(21) Appl. No.: 13/265,176

(22) PCT Filed: Apr. 21, 2010
(86) PCT No.: PCT/US10/31908

§ 371 Date: May 31, 2012

Related U.S. Application Data
(60) Provisional application No. 61/171,275, filed on Apr. 21, 2009.

(51) Int. Cl. C12M 1/34; G01N 21/64; G01N 33/569; G01N 24/00; C12Q 1/04; C12Q 1/06; G01N 21/25; G01N 27/00; G01N 33/53; C12Q 1/02

(52) U.S. Cl. 435/6.11; 435/287.2; 435/287.1; 435/288.7; 436/501; 435/7.2; 435/7.1; 435/29; 435/34; 435/39; 422/69

(57) ABSTRACT

In general, the invention features multiplexed devices, systems, methods, and kits for analysis of cells, particles, and other analytes on a porous membrane. Preferred devices detect, identify and quantify low levels of microorganisms in complex biological samples, such as blood. An exemplary device includes a housing having a fluid inlet that is in fluid communication with a plurality of channels, e.g., having substantially the same fluidic resistance. Each of the plurality of channels is in fluid communication with a reservoir containing reagents for analyzing cells, particles, or other analytes bound to particles, one or more substantially planar, porous membranes through which the cells or particles do not pass, and one or more outlets, wherein liquid flowing away from the inlet is divided between the plurality of channels and flows through the one or more membranes towards the outlet, and wherein the reservoir is disposed upstream of the one or more membranes.

### CROSS-REFERENCE TO RELATED APPLICATIONS

This application claims benefit of U.S. Provisional Application No. 61/171,275, filed Apr. 21, 2009, which is hereby incorporated by reference.

### BACKGROUND OF THE INVENTION

The invention relates to the field of analysis of cells, e.g., microorganisms, and other analytes, e.g. biomolecules.

The analysis, e.g., detection and identification, of pathogenic organisms, other cells, and biomolecules in the environment, foods, and clinical samples is important for human and animal safety, health, and welfare.

Exposure to pathogens in the environment is an increasing health problem because of demographic and other factors. Factors include population growth and urbanization, increased contamination of the atmosphere and water sources, spread of pathogens because of migration, international trade, and contact with animal populations. Bacterial and fungal contamination is a major concern for many industries including pharmaceuticals, cosmetics, eye-care products, and semiconductors. Suspected and actual incidents of bacterial contamination have led to costly and damaging product recalls.

Likewise, illness from food-borne pathogens is a significant health issue. It is estimated that contaminated food causes 76 million illnesses, 325,000 hospitalizations, and 5000 deaths annually in the United States (Mead, P S et al., 1999, Emerging Infectious Diseases 5 (5): 607-625).

According to the 2001 World Health Report from the World Health Organization, infectious and parasitic diseases are the second largest cause of death in the world. Methods for the detection and identification of pathogenic organisms are necessary for both the screening of individuals who are colonized with such organisms and thereby present a threat to themselves (during surgery for example) or to others (in nursing homes and hospitals) and for the diagnosis of infection in affected individuals.

Accordingly, there is a need for improved methods for the analysis, e.g., detection, identification, and enumeration, of pathogenic organisms, other cells, and biomolecules in a broad range of samples.

### SUMMARY OF THE INVENTION

In general, the invention features multiplexed devices, systems, methods, and kits for analysis of cells, particles, and other analytes on a porous membrane. Preferred devices detect, identify and quantify low levels of microorganisms in complex biological samples, such as blood.

Accordingly, in one aspect, the invention features a device including a housing having a fluid inlet that is in fluid communication with a plurality of channels, wherein each of the channels is in fluid communication with a reservoir containing reagents for analyzing cells, particles, or analytes bound to the particles, one or more substantially planar, porous membranes through which the cells or particles do not pass, and one or more outlets (e.g., that allow the passage of gas but not liquid), wherein liquid flowing away from the inlet is divided between the channels and flows through the one or more membranes towards the outlet, and wherein the reservoir is disposed upstream of, i.e., towards the inlet, the one or more membranes. In one embodiment, the reagents for analyzing bind to a target analyte bound to the particles.

Any device of the invention may further include at least one reservoir for the particles, wherein the reservoir is disposed between the inlet and one of the one or more membranes, and wherein the reservoir for the particles is in fluid communication with at least one of the plurality of channels. In any device of the invention, the housing may include a portion through which optical analysis of cells or particles on one of the membranes may occur. Any device of the invention may further include electrodes disposed adjacent to or on a membrane for electrical analysis of cells or particles. Any device of the invention may further include a magnetic resonance detector adjacent to a membrane for magnetic relaxation analysis of cells or particles. Any device of the invention may also include a reservoir for waste disposed between a membrane and an outlet. Any device of the invention may also include a reservoir containing liquid reagents disposed between the inlet and a membrane and separated from the channels by a valve. Any device of the invention may include a reservoir for containing a sample disposed between the inlet and a membrane and in fluid communication with the channels, optionally further including a reservoir containing liquid reagents separated from said sample reservoir by a valve.

Any device of the invention may further include a temperature sensor; a heating or cooling element; and/or a passive mixer or an active mixer element disposed between the inlet and a membrane.

In certain embodiments, the channels have substantially the same fluidic resistance. In other embodiments, the reservoir containing reagents for analyzing is disposed within a channel. Alternatively or in addition, a reservoir is separated from a channel by a valve.

Any device of the invention may also include a plurality of reservoirs of reagents for analyzing, where each reservoir is in fluid communication with at least one of the plurality of channels. For example, each of the plurality of reservoirs is in fluid communication with one, and only one, of the plurality of channels. Such reservoirs may be disposed with the channel or separated by a reservoir valve. When separated by a reservoir valve, the device may further include a plurality of channel valves that, when closed, prevent flow between the channel and the inlet

In certain embodiments, the reservoir of reagents for analyzing is in fluid communication with each of the channels, so that flow of reagents for analyzing away from the inlet is divided between the channels.

Membranes employed in the devices of the invention are preferably substantially nonfluorescent and/or resistant to degradation by alcohol, acid, or base.

Any device of the invention may also include a sample chamber and a liquid reagent chamber, where the sample chamber and the liquid reagent chamber are separated by a valve and are disposed between the inlet and the plurality of channels.

In certain embodiments, any reservoir or chamber of a device may be mechanically deformable chamber, where compression of the chamber expels its contents.

In a related aspect, the invention features a system having a receptacle for mating to (e.g., insertion of) any device of the invention and includes actuators for pumping fluids from the inlet of the device towards the outlet of the device; a temperature controller configured to interface with the device to control the temperature in at least a portion of said device; and a detector (e.g., an optical detector, electrical detector, or a magnetic relaxation or magnetic resonance detector) configured to interface with the device for analysis of cells, particles, or analytes bound to the particles on the membrane. The system may further include an active mixer element configured to interface with the device to mix two fluids between the inlet and the membrane. The system may also include comprising a reservoir for fluids and a pump to deliver fluids from said reservoir to said inlet of said device.

The invention also features a method of analyzing a sample (including cells, particles or analytes that bind to the particles) using a device of the invention. The method includes introducing the sample into the device, allowing the reagents for analyzing to contact the cells, particles, or analytes; capturing the cells or particles on the membrane; and analyzing the cells or particles on said membrane, e.g., for detection, enumeration, and/or identification. Preferred reagents for analyzing include probes (e.g., PNA, DNA, or LNA) for nucleic acids or antibodies. The methods may further include contacting the cells, particles, or analytes bound to said particles with reagents that bind to the probes or antibodies, resulting in signal amplification. Reagents for analysis may be labeled for optical, electrical, radioactivity, or magnetic detection. The reagents for analyzing may also include a plurality of reagents that are optically distinguishable and that bind to different cells, particles, or analytes bound to the particles.

The method may further include treating the sample with a liquid reagent prior to detection. The liquid reagent may be mixed with the sample by an active or passive mixer, as described herein. In one embodiment, the device includes a sample reservoir and a liquid reagent reservoir separated by a valve from the sample reservoir, and active mixing includes actuating the valve and transferring the liquid reagent to the sample reservoir or the sample to the liquid reagent reservoir. This process may also be repeated to move the volume of liquid between the two reservoirs until a desired level of mixing occurs.

In other embodiments, the method may further include treating the sample with a liquid reagent (e.g., diluent, lysis buffer, or particles having binding moieties to the analytes, e.g., biomolecules) in the device. Such treatment can be used to process a sample such that particles (e.g., to which analytes are bound) or cells of interest are retained on the membrane surface while the rest of the sample passes through. The samples may have a high content of interfering cells, as for example blood, that could clog the membrane. In some cases, the samples may additionally contain mucus, as for example bronchial samples, or protein, as for example urine samples, that can contribute to filter clogging. A combination of detergents and enzymes, as described herein, may be employed to lyse the blood cells and solubilize the cell debris, mucus and/or proteins while leaving microorganisms, such as bacteria and yeasts, substantially intact. In such embodiments, the device may include a passive mixer disposed so that the liquid reagent and the sample mix while flowing through the device and before contacting the membrane. Alternatively or in addition, the treating step may include actively mixing the sample with the liquid reagent. The treating step may also include raising the temperature of the liquid reagent and sample mix to a specified temperature (e.g., 37° C.) for a predetermined length of time. In certain embodiments, the device includes a sample reservoir and a liquid reagent reservoir separated by a valve from the sample reservoir, wherein the sample is introduced into the sample reservoir, the liquid reagent is stored in the liquid reagent reservoir, and the active mixing comprises actuating the valve and transferring the liquid reagent to the sample reservoir or the sample to the liquid reagent reservoir.

In other embodiments, the sample is contacted with the particles having binding moieties under conditions in which analytes, e.g., biomolecules, of the sample bind to the particles, which are then captured by the membrane.

The sample may also be contacted with control particles that are subsequently divided between the plurality of channels proportionally with the sample, wherein the control particles are captured by the membrane.

The reservoir of reagents for analysis may be disposed within each of the plurality of channels, with the reagents being released from the reservoir by flow of adjacent liquid. Alternatively or in addition, a reservoir of reagents for analysis is separated by a valve from each of the plurality of channels and actuating the valve results in contact of the sample with the reagents.

The invention further includes a kit including a device of the invention and a diluent, lysis buffer, hybridization buffer, or control particles.

Exemplary samples for use with the invention are a culture, an environmental sample, or a biological sample. Exemplary cells are microorganisms and/or those produced by a subject as a result of disease.

Specific uses of the invention are described in greater detail herein. For example, the invention may be employed in analysis of catheter related blood stream infection (CR-BSI) or yeast speciation. The multiplex nature of the invention also allows for the analysis of more than one organism per channel of the device. For example, a device may include four channels, each of which includes six, different reagents, which have at least three, different labels and which may be the same or different in each channel. Another device includes six channels, each of which includes six, different reagents, which have at least three, different labels and which may be the same or different in each channel.

The invention eliminates hands-on steps; allows multiplex testing on single samples; automates the scoring of the assay; increases the sensitivity of the test to allow direct analysis of low levels of analytes, e.g., biomolecules, and cells, e.g., microorganisms, in samples such as blood; provides enumeration of cells; and enables point-of-care and point-of-test applications. The invention has a sensitivity of at least 1-10 cfu/mL for yeast and 10-100 cfu/mL for bacteria in highly concentrated cellular samples such as blood. The invention also provides a wide dynamic range of sensitivity for various types of cells.

The analysis, e.g., detection and identification, of microorganisms according to the invention allows preventive and ameliorative actions to be taken and medical treatment decisions to be made. Further, the enumeration of cells, e.g., microorganisms, in a sample according to the invention may provide information necessary for decision making. In clinical microbiology for example, bacteria in urine, bronchial lavage, and other bodily specimens generally must be present in concentrations exceeding predetermined threshold levels in order to be considered true infections requiring clinical intervention. In the field of transfusion medicine, platelet concentrates are tested for the presence of bacteria. Concentrates with bacterial levels below 1000 cfu/ml are considered acceptable for transfusion use according to FDA guidelines. Quantitative analysis also allows temporal trends, spatial distributions, and chemical sensitivities to be determined.

Other features and advantages will be apparent from the following description, the drawings, and the claims.

By “reservoir” is meant a volume within a device in which reagents are stored, either in liquid, gel, or solid form, or in which a volume of fluid (e.g., sample or buffer) is contained. A reservoir may be a chamber within a device that is physically separated from a channel within the device and requires actuation to open to contact the reagents or volume of fluid with another portion of the device. Alternatively, a reservoir may be a constrained aliquot of reagents, e.g., dried or otherwise adhered to a channel wall, where liquid flowing through the device will contact the reservoir without actuation (other than that required for flow).

By “in fluid communication with” is meant allowing contact with fluid or flow of fluid between. Areas of a device separated by closed valves (e.g., a pinch valve or frangible seal) are in fluid communication with each other, as the term is used with the present invention.

By a “porous membranes through which target cells or particles do not pass” is meant a membrane having pores sized to prevent passage of a target cell or particle in the absence of lysis or disintegration.

By “through which optical detection may occur” is meant allowing transmission of the detected wavelengths of light, e.g., in the IR, visible, or UV spectrum.

By “passive mixing” is meant mixing requiring no energy input other than that required for fluid flow in an otherwise stationary fluidic structure.

By “active mixing” is meant mixing requiring the input of energy, e.g., magnetic or mechanical, other than that required for fluid flow.

### BRIEF DESCRIPTION OF THE DRAWINGS

FIGS. 1A-1C are schematic view of the bottom, cross section, and top of a device of the invention.

FIG. 2 is an expanded view of an alternative device of the invention.

FIG. 3 is a schematic view of the fluid channels, valves, and reservoirs of another device of the invention.

FIGS. 4A-4B are schematic views of the side and top of a device of the invention.

FIG. 5 is a schematic view of the fluid channels, valves, and reservoirs of the device of FIGS. 4A-4B.

FIG. 6 is a schematic view of a channel structure for mixing fluids.

FIGS. 7A-7B are schematic depictions of mixing of fluids in a channel shown in FIG. 6 and a device incorporating such a channel.

FIGS. 8A-8B are fluorescence micrographs of a membrane partially coated with aluminum, without and with backlighting.

FIG. 9 is a schematic depiction of a structure for supporting a membrane to ensure planarity.

FIGS. 10A-10D are graphs of the dissolution of reagents spotted in a channel.

FIGS. 11A-11F are schematic depictions of analysis reagents employed in a 4-channel device and the results obtained with various samples.

FIGS. 12A-121 are schematic depictions of a method of using the device of FIGS. 1A-1C.

FIG. 13 is a series of fluorescent micrographs showing the change in image as cells are contacted with hybridization reagent and then washed.

FIGS. 14A-14B are fluorescent micrographs showing the results of tests performed in 6-channel devices on samples containing different strains of yeast.

FIG. 15 is an exemplary block diagram of a system of the invention.

### DETAILED DESCRIPTION OF THE INVENTION

The invention provides devices for the multiplexed analysis of cells, e.g., microorganisms, and other analytes, e.g. biomolecules, associated systems, kits, and methods of their use.

Devices

In general, the invention provides multiplexed devices that split a sample into two or more aliquots for parallel or serial analysis in one or more flow channels. The devices further employ a porous membrane to separate biological cells or particles from dissolved or smaller components in a sample. The cells are analyzed, e.g., optically, after being contacted with one or more reagents that are stored on the device. The channels may be designed, e.g., by total length or cross-sectional area, to split the sample evenly or unevenly.

The multiplex nature of the devices allows for a single sample to be assayed for numerous different organisms in series or in parallel. Alternatively or in addition, the multiplex device allows for replicate assays to be performed on the same sample. On-device sample splitting allows for aliquots of the same sample to be assayed under different conditions in parallel. For example, each aliquot of a sample may be employed with different reagents, analysis techniques, or sample treatment conditions (e.g., temperature or chemical modification) or employ different porous membranes (e.g., to retain cells or other analytes of different sizes). Multiplexing can be increased through the use of multiple analysis techniques. For example, optical detection with multiple wavelengths would allow the analysis of more than one analyte in each flow channel through the use of reagents with distinguishable colors for different analytes.

Uneven sample splitting may be employed when different types of cells are expected to be present in different numbers in a sample. Unequal sample splitting allows the delivery of an appropriate volume for assay; for example, a smaller volume is needed for cells present in larger numbers than that needed for cells present in smaller numbers. Uneven sample splitting also allows a single sample to be assayed for the same type of cell with a range of sensitivity, e.g., where the number of cells potentially present in a sample is highly variable.

The invention will now be described in greater detail with reference to specific examples.

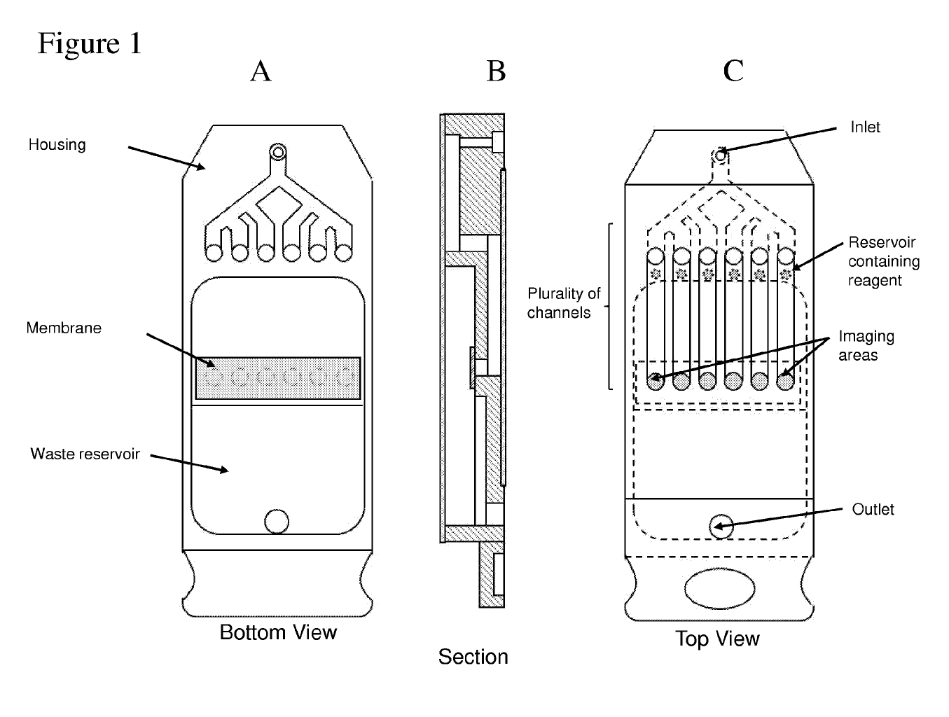

FIGS. 1A-1C shows a device of the invention. The device includes a housing of a molded body that is sealed with transparent tape. In this embodiment, the device is designed so that only one molded part is required to define the plurality of channels. A porous membrane is adhered to the body as indicated. The single use cartridge contains all reagents for analysis and stores the generated waste. This device includes a single inlet through which the sample is loaded and a structure that splits the sample and directs a part of the sample volume into each of multiple, e.g., 6, channels. Each of these channels includes a reservoir of reagents that are dried or otherwise deposited on the channel surface and that are for analyzing cells, particles, or other analytes. The reagents interact with, e.g., bind, components in the target cells or particles (e.g., bound analyte) and allow analysis, e.g., by fluorescence imaging. The device allows for analysis, e.g., via imaging, of distinct areas of the membrane for each channel.

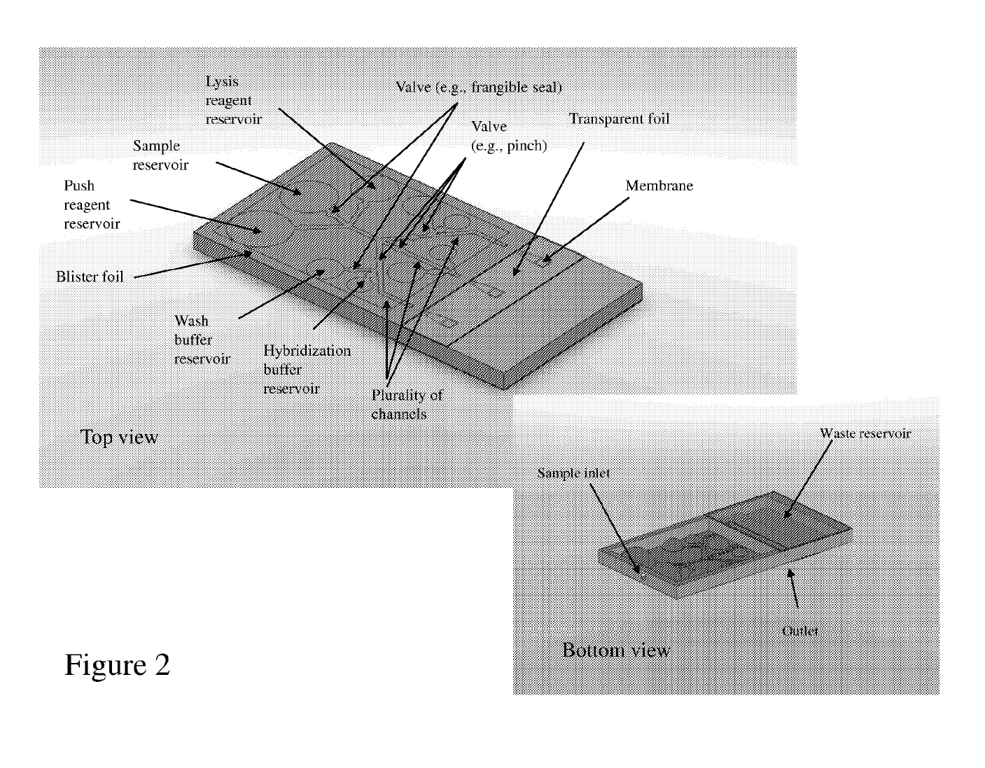

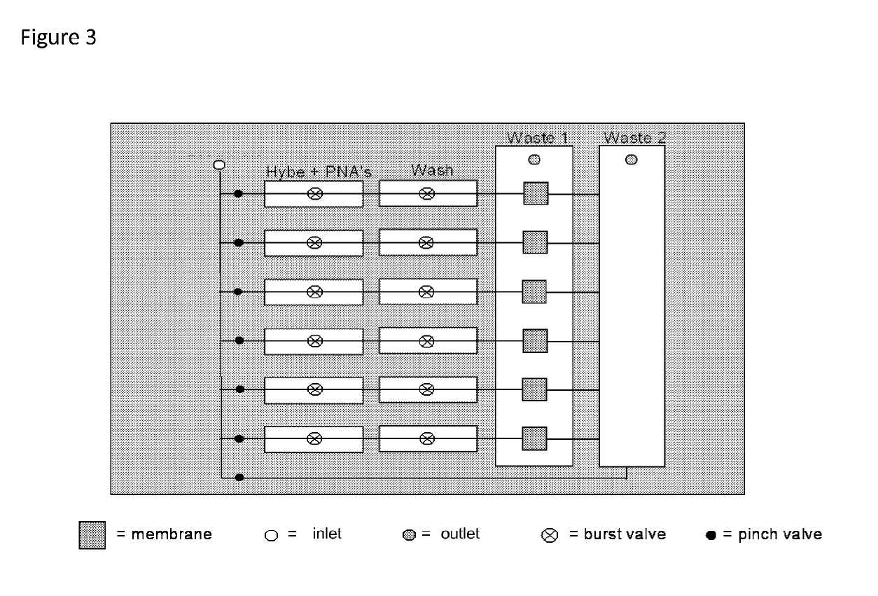

An alternate device is shown in FIGS. 2 and 3. These devices include blister packs, i.e., reservoirs, to store liquid reagents for use in the assays. Blister packs, as are known in the art, are structures widely used for storage of prescription and non-prescription pills and capsules. Blister packs are may also be used for the storage of fluids, as described for example in U.S. Pat. No. 5,374,395. Blister packs can be made by cold forming aluminum and polymer laminates to create cavities and thermally bonding two or more layers to form sealed chambers and flow passages. The thermal bonding can be done so that the bond is frangible in particular areas. A frangible bond, which acts a valve, is designed to give way when sufficient pressure is generated in the contents of a blister. The presence of a frangible bond gives the blister pack long storage life in sealed form while allowing the content to be expelled into the flow passages of the device. Other chamber configurations for storing liquid reagents may also be employed (e.g., a rigid chamber whose contents are expelled by a piston, pump, or other force). The devices of FIGS. 2 and 3 may include a housing having single molded body that is sealed with transparent tape and blister foil. Each of the plurality of sample channels in these devices is connected to a reservoir that stores the reagents for analysis (e.g., Hybridization buffer (Hybe) with peptide-nucleic acids (PNAs)) and to a porous membrane for separation of cells, particles, or other analytes (e.g., bound to particles) for analysis. The reservoirs are isolated from the channels by a valve, e.g., a burst valve, such as a frangible seal, that remains closed until actuation. The devices may also include one or more reservoirs for other reagents, e.g., push reagent, lysis reagent, and wash buffer as shown. This devices includes valves, e.g., pinch valves, that are used to isolate each of the channels from the rest of the device. These valves are closed to prevent backflow when the analysis reagent reservoirs and wash buffer reservoirs are actuated. A device of the invention may also include a channel that does not pass through a membrane, as shown in FIG. 3, allowing for an alternate route for pressure release or for overflow of sample or reagent.

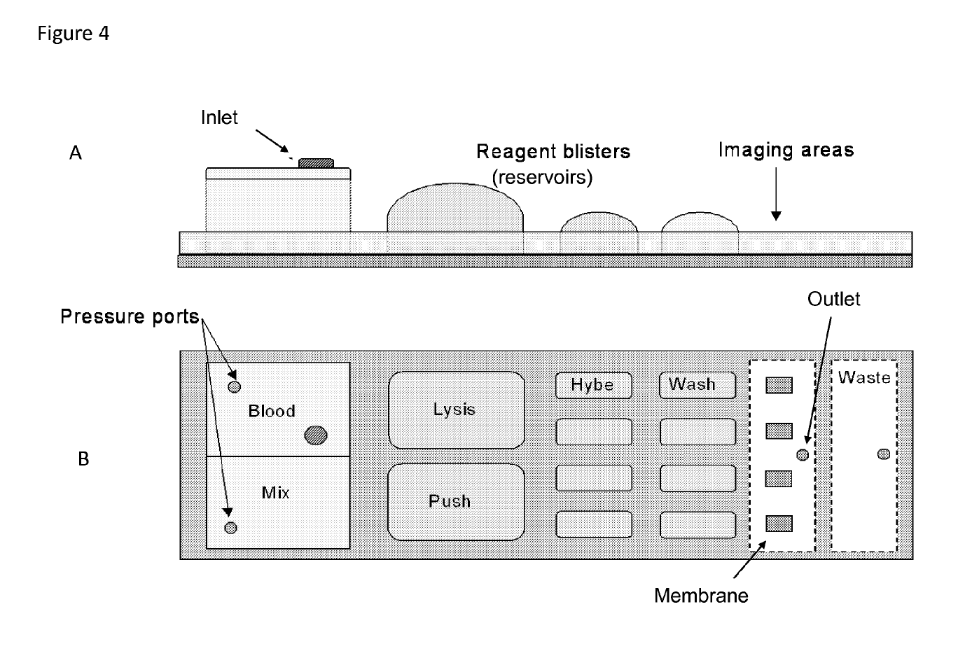

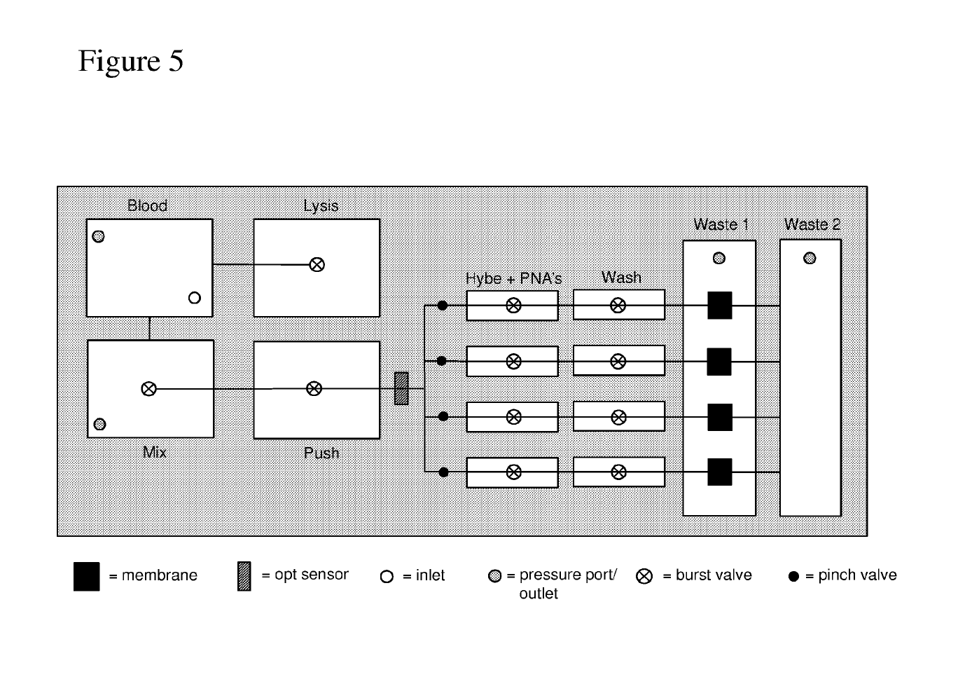

Another device is shown in FIGS. 4A-4B and 5. This device also employs reservoirs for containing reagents (hybridization buffer), and optionally a wash buffer. In addition, this device includes reservoirs for lysis buffer and a “push” buffer. Again, these reservoirs are isolated until actuation of a valve, e.g., a burst valve. This device allows on-device lysis of blood cells (or other sample treatment prior to contact with the analysis reagents). The sample may be introduced into a sample, e.g., blood, chamber and mixed with the lysis or other reagent. This mixture may then be moved between two reservoirs, e.g., the mix and sample reservoirs, on the device to ensure complete mixing of the two fluids. In this example, fluid can be moved between reservoirs using pressure ports, where positive pressure in one reservoir moves the liquid to the other reservoir. Alternatively, negative pressure can be used to pull a liquid from one reservoir to another. Fluid in the “push” reservoir may also be employed to ensure that the entire sample is pushed through the membrane. This device is capable of storing all fluids and reagents necessary to complete an assay, once a sample is loaded. Reagents can be stored in the device in dried form. A solution of the reagent may be spotted on a wall of one or more channels in the device and dried in the manufacturing process. Alternately, beads of dried reagent can be incorporated into the device during manufacture. The use of a fluid reservoir and the ability to move fluid back and forth between reservoirs may also be employed in combination with dried reagents that are stored in the device and that can be dissolved on-device at the appropriate time to perform an assay.

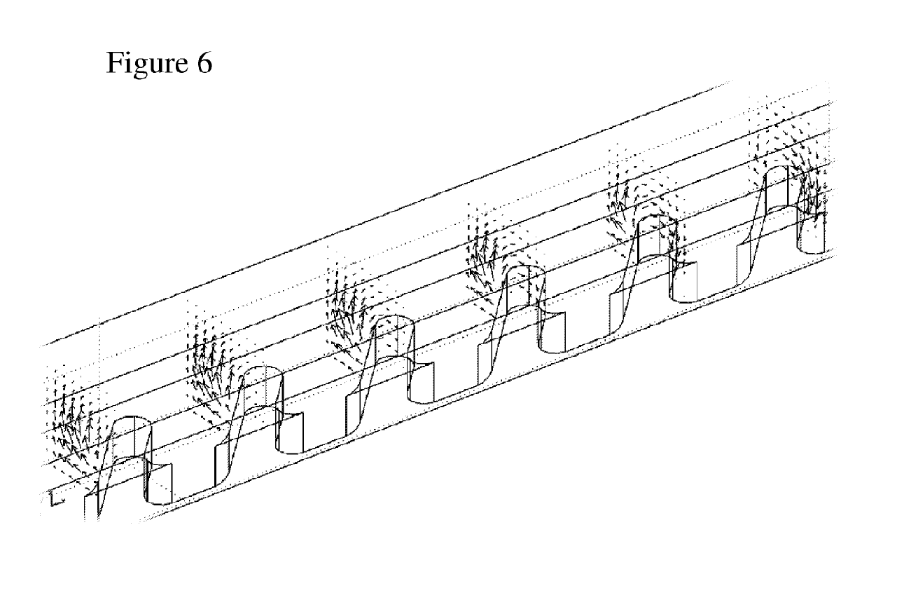

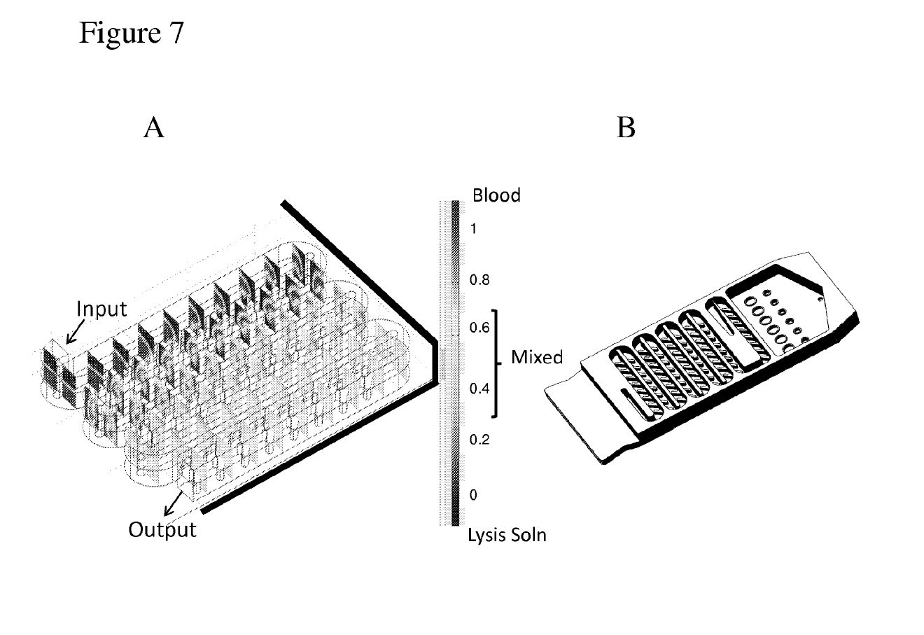

In addition to the use of multiple chambers for mixing two fluids, any other suitable mixing technique maybe employed. A passive mixing technique is illustrated in FIG. 6. In this technique, the channels include structures that passively mix two fluids (Stroock et al. Anal. Chem. 2002; 74:5306-5312) using chaotic mixing (Leong et al. Phys Rev Lett. 1990; 64:874-877). Such a channel may be incorporated in a serpentine design as shown in FIGS. 7A-7B. Active mixing techniques may also be employed, e.g., use of a mechanical or magnetic stir bar, mechanical shaking, or ultrasonic mixing. In such embodiments, an element of the active mixer may be included in the device, e.g., in the sample reservoir, lysis reservoir, mix reservoir, or a channel. For example, the device may include a magnetic stir bar or a mechanically rotated component that is actuated by other elements in the device or mated to the device, such as a rotating magnet or rotating motor for mechanical stirring.

Devices of the invention may be manufactured out of any suitable material. For example, the housing of the device is fabricated in cyclic olefin copolymer (e.g., Topas 5013 or Zeonex) by hot embossing and sealed with polyolefin tape (3M 9795R). This tape incorporates a silicone-based adhesive that is suitable for devices in which alcohol-containing buffers are used. Other polymers such as cyclic olefin polymer, polycarbonate, or polymethyl-methacrylate may be used. In addition to hot embossing, other manufacturing techniques such as injection molding may be used.

Devices of the invention also include one or more outlets to allow liquids or gas to escape during pumping of fluids. Preferably, the device includes a reservoir for storing waste liquids and reagents, e.g., to allow for containment of potentially biohazardous waste and ease of disposal. The outlet may be covered with a suitable material to prevent (or substantially retard) the passage of liquids but allow gas to escape.

Membranes. Suitable membranes for separating cell, other particles, and other analytes (e.g., bound to particles) from fluids and smaller debris are known in the art. Typically, such cells or particles will have a size of greater than 0.1 μm. The membrane may be adhered to the device using adhesives, thermal bonding, ultrasonic welding, laser welding, or compression fitting. The membranes for the individual channels may be provided as a single element (e.g., a single strip of material spanning all channels) or multiple elements in the device. When the membranes are single elements, the material used preferably has no lateral porosity, so that individual aliquots do not mix in the device.

Exemplary membranes are shown in Table 1. Track-etch membranes have cylindrical pores created by etching through a film of polycarbonate (or polyester). Anopore aluminum oxide membranes are formed by electrolytic oxide formation on aluminum followed by dissolution of the aluminum. These membranes are flat and have high porosity. The substrate is brittle, which can be a disadvantage. Black Nylon is a depth filter with carbon particles incorporated among the nylon strands. It is not as flat as the other filters.

TABLE 1

| | | Fluores- | | Flatness & | |
|---|---|---|---|---|---|
| | Vendor | cence | Porosity | Rigidity | Bonding |
| Track-etch | GE | Moderate | 9.4% | Flat | UV |
| Poly- | Nucleonics, | | | Compliant | adhesive |
| carbonate | Sterlitech | | | | Thermal |
| Anopore | Whatman | Low to | 25-50% | Flat | UV |
| Aluminum | | Moderate | | Rigid | adhesive |
| Oxide | | | | | |
| Black | CUNO (3M) | Low | absorbent | Textured | Thermal |
| Nylon | | | | Compliant | Ultrasonic |

Preferred characteristics of the membrane include pore size: 0.4 to 0.8 μm; pore density: >3×10⁷ per cm²; flat and smooth surface; bondable to plastic by any suitable method; low background fluorescence; and sufficiently low absorption of incident light so that heating of the membrane during optical detection is not excessive. Membranes are also preferably resistant to nonspecific binding of the analysis reagents. An area of 7 mm² is typically sufficient for analysis of cells in each channel of a 6-channel device processing 1 mL of sample, such as blood.

Membranes may be treated with dyes (e.g., irgalan black) or coated for reduced background fluorescence and improved imaging. Irgalan black-dyed polycarbonate membrane filters are commercially available from several suppliers (Sterlitech, SPI Supplies, and others). Exemplary coatings include carbon black, electroless nickel, sputtered gold or gold-palladium, and evaporated aluminum (Durtschi et al. Journal of Medical Microbiology 2005; 54:843-850 and Nishimura et al. Fisheries Science 2006; 72:723-727).

Specific membranes include a track-etch polycarbonate membrane with 0.6 μm pore size, 9 μm thick, and 3×10⁷ pores/cm²; PVP (polyvinyl pyrrolidone) coated with 50 nm aluminum coating; and a track-etch polycarbonate membrane with 0.4 μm pore size, 10 μm thick, 1×10⁸ pores/cm², PVP-free, coated with 50 nm aluminum coating.

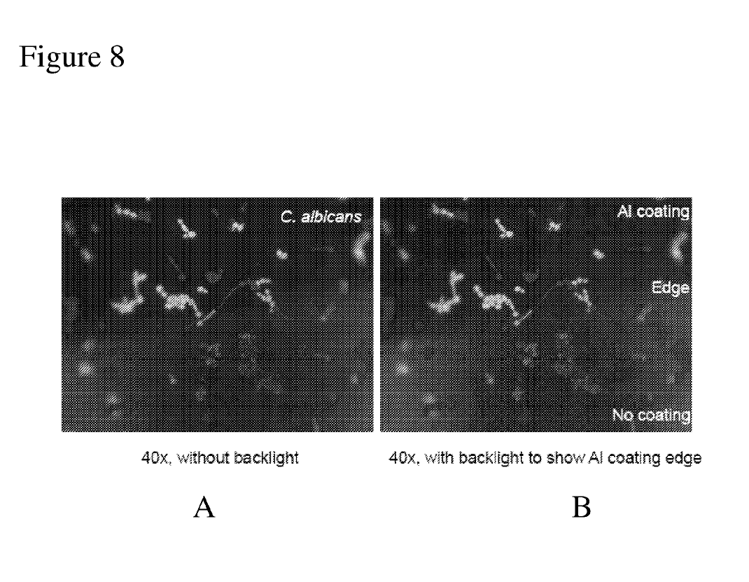

Aluminum coating has significant advantages, including low background fluorescence; low absorption of excitation light and therefore little heating, allowing rapid imaging; smooth surface; no leaching; open pores; and scalable, cost effective fabrication. FIGS. 8A-8B show micrographs of cells on coated and uncoated membranes.

Aluminum is reactive at high and low pH. An aluminum coating can be attacked by basic or acidic media. If basic or acidic media are used in the assay, the aluminum can be protected from attack with an overcoating of SiO₂. A track-etch polycarbonate membrane with 0.8 μm pore size, 9 μm thick, and 3×10⁷ pores/cm² coated with 50 nm aluminum and 50 nm of SiO₂ is suitable for use with basic media.

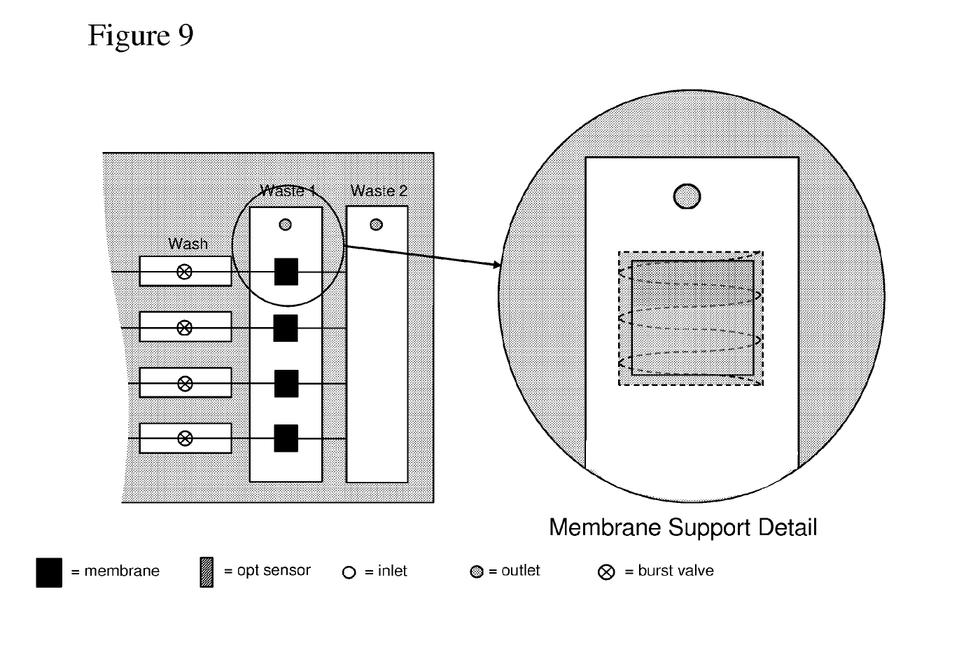

For optical detection, the membrane is preferably substantially planar. Supporting structures may be employed to maintain planarity, as shown in FIG. 9. Minimization of the amount of liquid between the membrane and imaging optics, e.g., by minimizing the channel depth, is also desirable. Optical access to the membrane surfaces is provided through a transparent layer or wall. This layer may be the sealing tape, a wall of the molded housing, or a window of a transparent material such as glass that has been attached in a sealed manner to the device. An optical detector may also be placed inside a device and be not visible from the exterior.

Additional Components:

Devices of the invention may further include additional elements, e.g., for use in sample introduction, movement, analysis, and storage. For example, a device of the invention may include a reservoir for receiving a sample and further include a receptacle in the sample chamber for receiving sampling implements, such as swabs, pipettes, or syringe needles. Examples of such receptacles include septa and openings in the device. Any opening could be closed after the sampling instrument has introduced the sample, or the sampling instrument could be sealed to the device via a septum or gasket.

A device of the invention may also include one or more optical sensors, e.g., as shown in FIG. 5. A sensor may be employed to determine when a particular amount of a fluid, e.g., blood or a fluid containing an optically detectable reagent, has passed through the device. Other types of sensors, e.g., electrodes or temperature sensors, may also be employed for this purposed.

Devices of the invention may also include heating elements, e.g., resistive heating elements, either embedded in the device or disposed adjacent the device to control the temperature. Temperatures sensors, e.g., thermistors or thermocouples, may be employed to monitor the temperature and/or provide thermostat control.

Devices of the invention may also include elements for analysis, including optical elements, e.g., filters, lenses, and light sources (e.g. LEDs) and electrodes, e.g., for conductivity, voltammetry, or amperometry.

It will also be understood that devices of the invention may be constructed in variations of those elements described herein. Devices may also include two or more independent inlets connected to an independent plurality of channels, e.g., to assay two or more samples or aliquots of the same sample on the same device. A device may also employ more than one type of analysis, either simultaneously or sequentially; for example, a sample may be assayed optically and electrically. In such a configuration, the device may include multiple analysis reagents for each method, or one or more of the methods of analysis may rely on an intrinsic property of the sample. Devices may include different types of reservoirs; for example, a single device may employ reagents adhered to a channel wall and analysis reagents stored in a chamber sealed with a valve. Devices may include any number of channels for sample splitting, and each channel may employ the same or different method of analysis and/or analysis reagents.

System

The invention also includes a system for analysis and/or actuating the devices described. The system includes a receptacle for mating to the device, e.g., by insertion. Depending on the type of device employed, the system may include fluid reservoirs and pumps for delivery and movement of reagents and/or sample through the device. Alternatively or in addition, the system includes actuators for valves on the device. For burst valves, such actuators may apply mechanical pressure sufficient to burst the seal on the valve. Pinch valves are also actuated by mechanical pressure applied to the pinch point. Other valving schemes are known in the art. Compression of reservoirs containing fluids in the device may also be used to pump fluids in the devices, obviating the need for separate pumps.

The system also includes a detector, usually an optical imager. If an optical imager is used, it is typically configured for fluorescence detection, although other photometric detection is possible, e.g., absorbance, phosphorescence, turbidometry, and chemiluminescence. The imager may include a light source, e.g., a light emitting diode (LED), laser, or broadband source such as an arc or filament lamp, appropriate for the optical signal being detected. An exemplary light source uses three LEDs: Blue (457 nm), e.g., for fluorescein or Alexa 488; Green (525 nm), e.g., for Tamra or Alexa 532; and Red (640 nm), e.g., for Cy5 or Alexa 647. LED's with high output are available from Luminus Devices, Inc (Billerica, Mass.). The imager also includes an objective lens. Exemplary objective characteristics are 20× magnification 0.45 numerical aperture (NA) and 1.25 mm field of view (FOV). More preferably, a 10× magnification, 0.45 NA objective (Nikon Inc, Melville, N.Y.) can be used with imaging lenses that provide an overall magnification of 17.5× and a 2.5 mm FOV. The imager may also includes a photosensitive component, e.g., a photodiode, charge coupled device (CCD) array, or photomultiplier tube (PMT). The optical system of magnification 17.5× can be combined with a CCD camera with 7.4 micron pixels formatted as 4872×3248 pixels (DVC, Austin, Tex.) to image the 2.5 mm FOV. Optical filters and lenses may also be employed as is well known in the art. Particularly suitable fluorophores and filters are shown in Table 2.

TABLE 2

| Fluorophore | Cube | Vendor | Ex | Dichroic | EM |
|---|---|---|---|---|---|
| Fluorescein | XF 100-2 | Omega | 475AF40 | 505DRLP | 535AF45 |
| Tamra | CY3- | Semrock | 531/40 | FF562 | 593/40 |
| | 4040B | | | | |
| Cy5 | CY5- | Semrock | 628/40 | 660-DiO1 | 692/40 |
| | 4040A | | | | |

Non-optical methods such as electrochemical methods can be used by incorporating electrodes into the device. The electrodes then connect to circuitry in the system. Examples of such measurements include amperometry, cyclic voltammetry, or conductivity. Electrochemical analysis of pathogens in urine specimens has been achieved using gold electrodes on a plastic substrate in combination with DNA capture and analysis probes (Liao et al. Journal of Clin Microbiol 2006; 44:561-570). The analysis of specific oligonucleotides in blood and other samples has been achieved with alternating-current voltammetry of redox-labeled DNA stem-loop probes on gold electrodes coated with a self-assembled alkanethiol monolayer (Lubin et al. Anal. Chem. 2006; 78 5671-5677).

Magnetic detection or detection of radioactivity can also be used. For example, magnetic relaxation measurements can be used to analyze pathogens, e.g., Mycobacterium avium spp. Paratuberculosis, based on the aggregation of magnetic nanoparticles (Nano Lett., 2007; 380-383).

The systems may also include a heating and cooling system for temperature control of the device, e.g., from 20-80° C. Heating and cooling may be effected by elements that are part of the device or that contact the device when inserted in the system. Heating and cooling may be effected by Peltier elements, resistive heating elements, heat sinks, cooling fans, or heated/cooled circulating fluids. Heating and cooling may also be effected by heated or cooled air flow around the device when inserted in the system.

The system may also include software for the analysis, e.g., detection and/or enumeration, of cells, particles, or analytes (e.g., bound to particles) on the membranes of the device. The software may also be employed to distinguish between different types of cells, particles, or analytes (e.g., bound to particles) based on color, shape, size, brightness, or secondary morphology (e.g., clustering). Such software is commercially available from a number of vendors, e.g., Metamorph (MDS) and Image Pro (Media Cybernetics) or can be created using mathematical software such as MATLAB (Mathworks). The nature of the software may also depend on the analysis method employed.

Other components may be provided. For example, a bar-code reader may be included for scanning identifying labels on devices and patient identifiers associated with samples. Such bar-code readers may be built into the system or be an external, hand-held type that connects to the system via a cable or wireless connection. A printer may be used for generating a printed readout that can be incorporated into a patient chart or record. A system may also include hardware and software for connecting to host computers in the facility, such as a hospital information system.

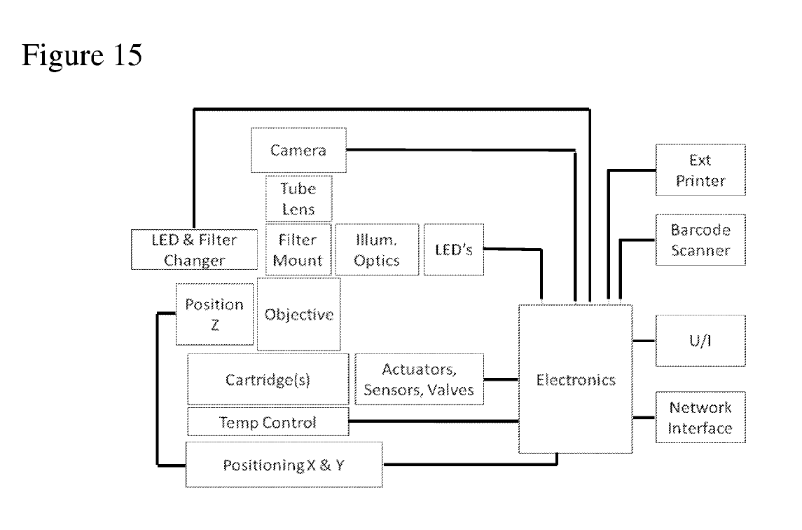

The elements of a system may be housed together in a single unit or may be separate components. In addition, although described as part of the system, as opposed to the device mated to the system, elements required for analysis, fluid movement, and temperature may be integral to the device, the system, or divided between the two, as described herein. An exemplary system block diagram is shown in FIG. 15.

Methods

The devices of the invention are employed to analyze cells, particles, and other analytes (e.g., bound to particles) in various samples. The steps employed in the methods typically include passing the sample, which may be pre-treated, through the device so that cells, particles, or other analytes are deposited on the membrane. The cells, particles, or analytes (e.g., bound to particles) are contacted with a reagent for analysis and imaged or otherwise analyzed as described herein. A washing step may also be employed to remove any analysis reagent that would interfere with accurate measurement.

In some methods, cells are lysed to release their contents. Target biomolecules, such as DNA, RNA, proteins, lipids, and complexes thereof, may then be captured on particles, e.g., beads, provided in the device. Analytes in a sample may also be bound to particles prior to introduction into the device. The particles are typically surface functionalized with binding moieties, e.g., antibodies or sequence specific probes for nucleic acids, designed for the capture of the target analytes. Such particles are well-known in the art, for example, latex beads, silica heads, and paramagnetic beads. Following the capture step, the particle mixture is passed through the device and deposited on the membrane. Analysis may then occur as with cells. For example, the particles may be fluorescent. Particles with different binding moieties may emit different colors as fluorescence as in the Luminex xMAP system. Such particles can then be mixed and distinguished by the emitted color. This allows multiple analytes to be analyzed in the same assay. Binding moieties labeled with reporter fluorophores may be used for analysis, e.g., detection and quantification of the analytes. The reporter fluorophores are of a different color than the particles if the particles are fluorescent.

The methods can also be used to analyze any type of cells. For example, the methods may be used to identify organisms from a culture, an environmental sample, e.g., air, water, soil, or industrial sample, or a biological sample, e.g., blood, plasma, serum, bronchoalveolar lavage, endotracheal aspirates, sputum, urine, cerebrospinal fluid (CSF), and lymph. The methods may be used to analyze plant cells, animal cells, bacteria, fungi (e.g., yeasts), and protists, e.g., to identify a particular species of organism or other classification, e.g., bacterial, fungal, or protist. Exemplary uses are for identifying infectious organisms, e.g., the species of yeast or bacteria in a blood culture and for detecting and identifying catheter related blood stream infection (CR-BSI). Yeast analysis can be used to identify C. albicans, C. glabrata, C. krusei, C. parapsilosis, and C. tropicali, for example. The methods can also be used to distinguish S. aureus vs. coagulase-negative Staphylococci (CNS); E. faecalis vs. other Enterocococci spp.; E. coli and K. pneumoniae (EK) vs. P. aeruginosa; C. albicans vs. other Candida species; and Gram+ and Gram− organisms (optionally) in blood cultures or CR-BSI. The methods of the invention may also be used to assay cells from a patient, e.g., to diagnose a disease state. Such cells include cancer cells, red and white blood cells, progenitor cells, stem cells, fetal cells, epithelial cells, endothelial cells, mesenchymal cells, and platelets. Particulate cellular organelles, e.g., nuclei, chloroplasts, and mitochondria, may also be analyzed with or without binding to other particles for separation.

Preferred analysis agents are labeled nucleic acid binding probes, e.g., PNA FISH probes as described in WO 2005/121373, which is hereby incorporated by reference, DNA, and LNA probes. The probes may be labeled with a variety of detectable tags including fluorophores, enzymes (e.g., alkaline phosphatase or horseradish peroxidase), electrochemically active labels, magnetic particles, biotin, and haptens. Other analysis reagents include labeled antibodies, aptamers, and intracellular dyes. Reagents for immunoassays may also be employed, e.g., antibody-enzyme conjugates as with ELISA.

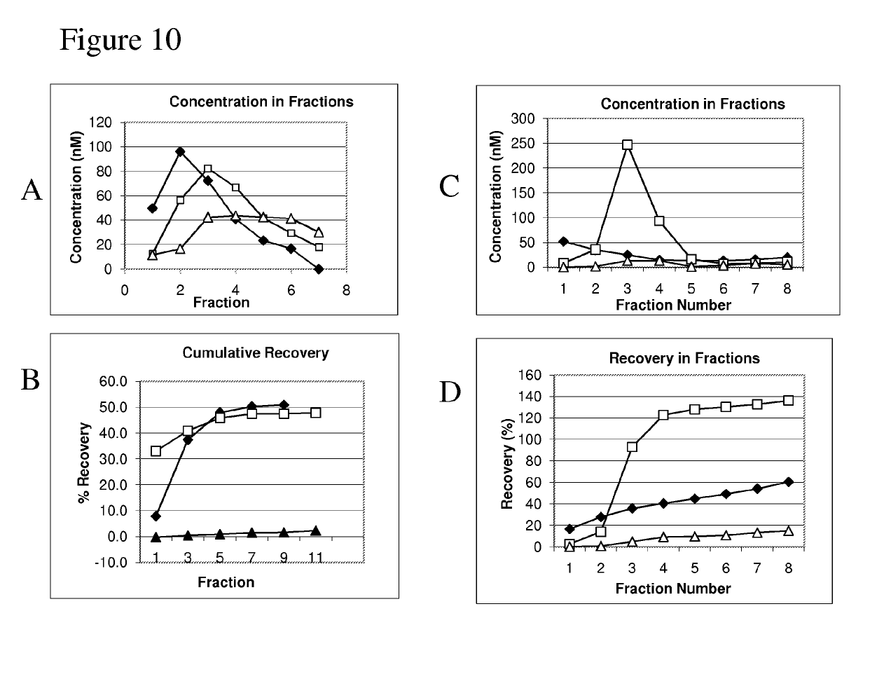

Reagents may be stored on the devices in reservoirs, e.g., sealed chambers or dried or gelled locations in channels. For reagents deposited on channels, the geometry and matrix determines the time necessary to dissolve the reagents. FIG. 10A shows the effects of geometry on dissolution. As shown in the figure, deposition on a flat surface (diamonds) results in faster release than deposition in a well (squares, triangles). The ratio of the width to the depth of a well also affects the rate of release, with a narrower, deeper well (triangles) resulting in a slower release. FIG. 10B shows the effects of the matrix on dissolution. Sucrose (squares) and no matrix (diamonds) result in more rapid release than a dextran sulfate matrix (triangles). FIGS. 10C-D show the effects of dissolution rate for different matrices: 360 kDa polyvinyl pyrrolidone (PVP) (squares), dextran sulfate (DS) (triangles), and mannose (squares). PVP results in a faster release than either mannose or dextran sulfate. Additional matrix materials include polyvinyl alcohol and polyethylene glycol.

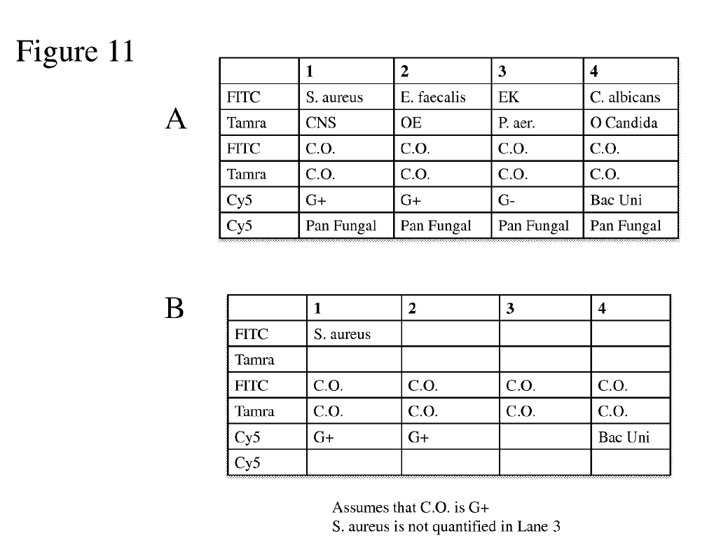

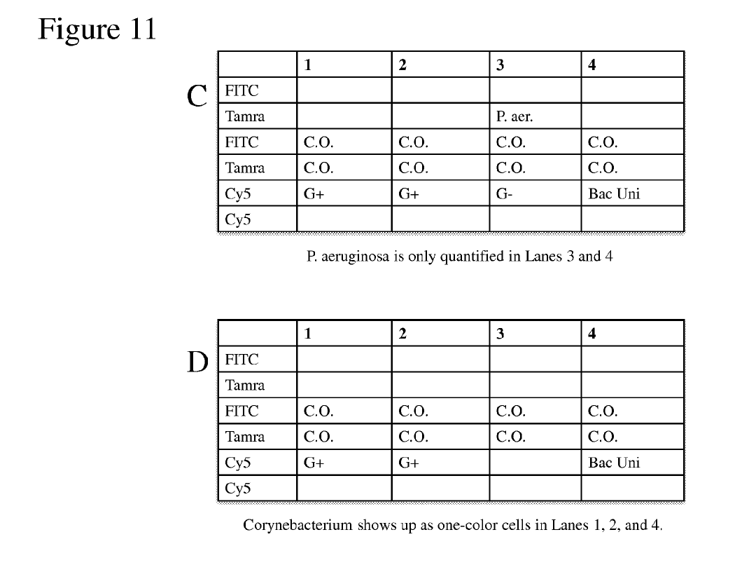

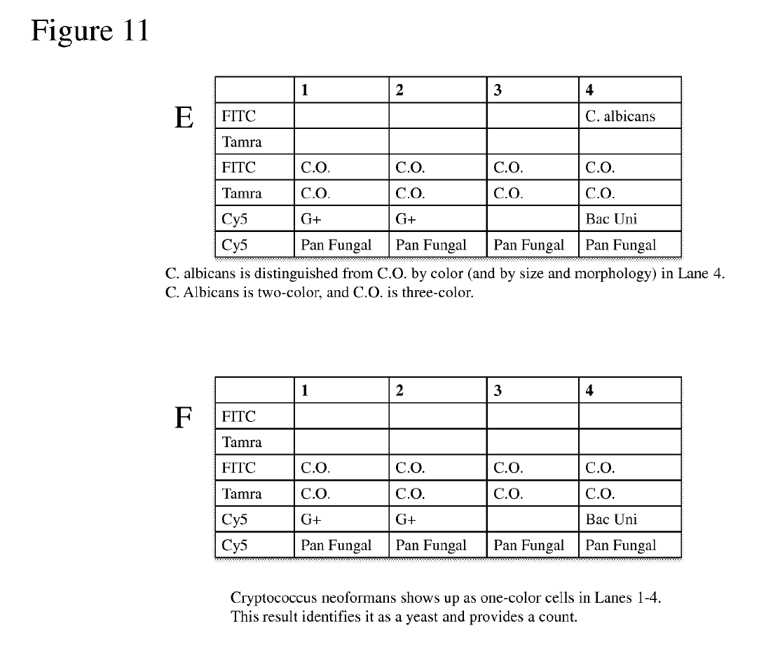

Optical detection may be monochromatic or multicolor. Sets of fluorophores that may be used include FITC (or Alexa 488)/Tamra/Cy5 (or Alexa 647); FITC/Texas Red/Cy5; and Alexa 405/FITC/Texas Red. In principle, the use of 3 colors allows us to encode 7 entities in each channel of the device. An example of this is shown in FIGS. 11A-11F. FIG. 11A shows lane assignments for a four-lane device. Lane 1 includes green-labeled (FITC) reagents for S. aureus and control organism (C.O.); red-labeled reagents (Tamra) for CNS and C.O.; and purple-labeled reagents (Cy5) for gram positive (G+) and pan fungal. Lane 2 includes green-labeled reagents for E. faecalis and C.O.; red-labeled reagents for other Enterococci (OE) and C.O.; and purple-labeled reagents for gram positive (G+) and pan fungal. Lane 3 includes green-labeled reagents for E. coli+K. pneumonia (EK) and C.O.; red-labeled reagents for P. aeruginosa (P. aer) and C.O.; and purple-labeled reagents for gram negative (G−) and pan fungal. Lane 4 includes green-labeled reagents for C. albicans and C.O.; red-labeled reagents for other Candida spp. (O. Candida) and C.O.; and purple-labeled reagents for universal bacterial (Bac Uni) and pan fungal. FIG. 11B shows the results for a sample including S. aureus. In this assay, in lane 1 S. aureus cells will be stained green and purple, and C.O. will be stained green, red, and purple; in lane 2 S. aureus cells will be stained purple, and C.O. will be stained green, red, and purple; in lane 3 S. aureus will not be stained, and C.O. will be stained green and red; and in lane 4 S. aureus will be stained purple, and C.O. will be stained green, red, and purple. The results show that S. aureus is present in the sample with no yeast or other bacteria in the sample. FIG. 11C shows the results for a sample including P. aeruginosa. In this assay, in lanes 1 and 2, P. aeruginosa will not be stained, and C.O. will be stained green, red, and purple; in lane 3 P. aeruginosa will be stained red and purple, and C.O. will be stained red and green; and in lane 4 P. aeruginosa will be stained purple, and C.O. will be stained green, red, and purple. The results show that P. aeruginosa is present in the sample but not yeast or other bacteria. FIG. 11D shows the results for a sample including Corynebacterium. In this assay, in lanes 1, 2, and 4 Corynebacterium will be stained purple, and C.O. will be stained green, red, and purple; and in lane 3, Corynebacterium will not be stained, and C.O. will be stained green and red. The results show that an unidentified gram positive bacterium is present in the sample, and this organism can be counted. FIG. 11E shows the results for a sample including C. albicans. In this assay, in lanes 1 and 2, C. albicans will be stained purple, and C.O. will be stained green, red, and purple; in lane 3, C. albicans will be stained purple, and C.O. will be stained green and red; and in lane 4, C. albicans will be stained green and purple, and C.O. will be stained green, red, and purple. The results show that C. albicans is present in the sample but not bacteria or other yeasts. FIG. 11F shows the results for a sample including Cryptococcus neoformans. In this assay, in lanes 1 and 2, Cryptococcus neoformans will be stained purple, and C.O. will be stained green, red, and purple; in lane 3, Cryptococcus neoformans will be stained purple, and C.O. will be stained green and red; and in lane 4, Cryptococcus neoformans will be stained purple, and C.O. will be stained green, red, and purple. The results show that a yeast is present in the sample but not bacteria, and this yeast can be counted.

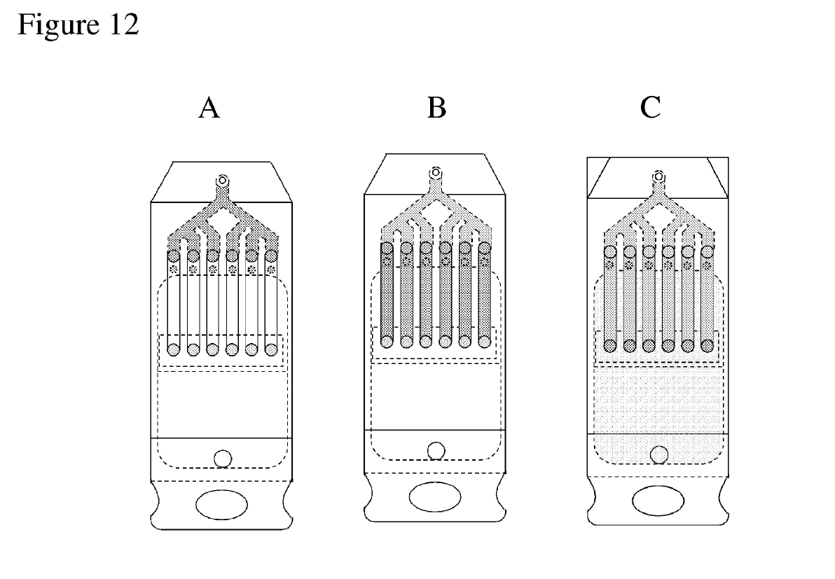

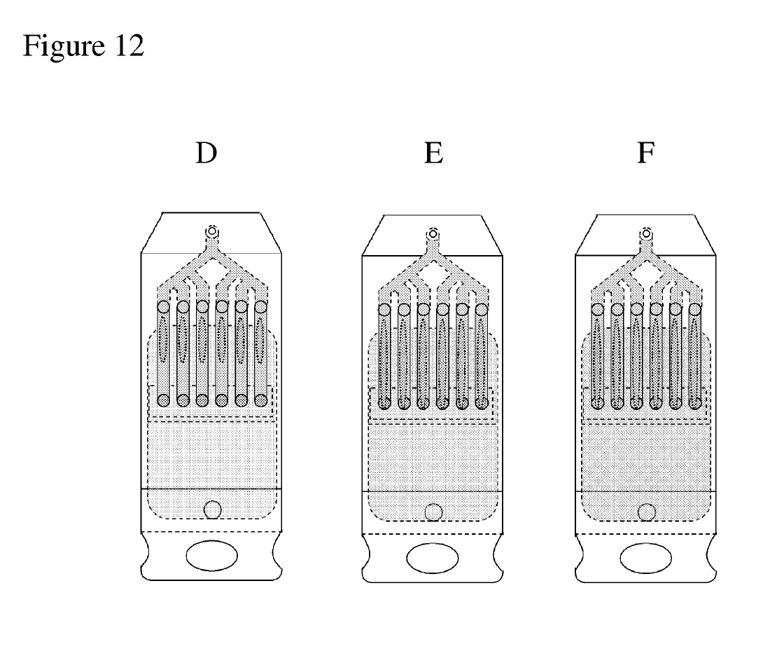

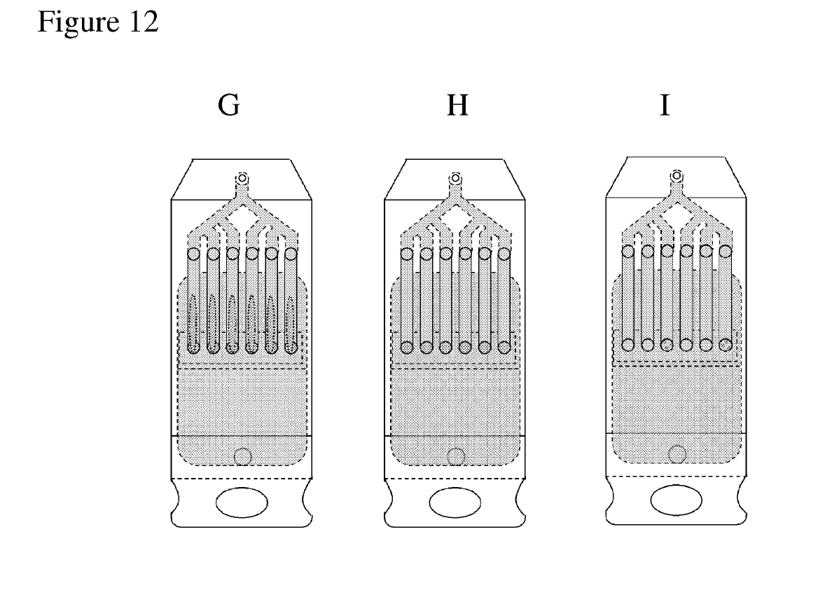

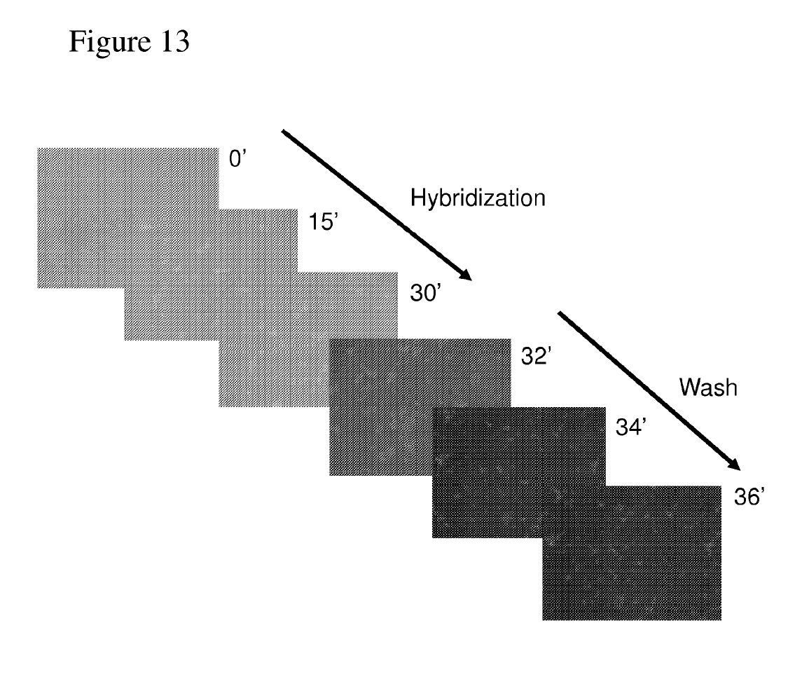

FIGS. 12A-12I show a schematic of the method using the device of FIGS. 1A-1C. In step (a), sample, e.g., 100 μL-1 mL, is loaded, e.g., manually via pipette. The device is then connected to a system in step (b), and liquid, e.g., 600 μL/min of hybridization buffer, is pumped through the device, e.g., for 3 minutes. In step (c), pumping of liquid continues; for example, the rate of flow is decreased to 20 μL/min, and the device is heated to 55° C. In steps (d)-(f), pumping of liquid continues resulting in the release of reagents for analysis (e.g., PNA FISH reagent) deposited in each of the plurality of channels (illustrated as elongating ovals). These steps may occur, for example, over 27 minutes. In steps (g)-(h), pumping of liquid, e.g., 300 μL/min for 5 minutes, continues resulting in washing away unbound reagent from analytes. Stained cells or particles are then imaged, e.g., at <35° C. (step (i)). Images of this sequence of events are shown in FIG. 13.

Exemplary process steps are described in the examples. These steps may be employed in any method of the invention.

Sample Preparation

Samples may or may not be pre-treated prior to delivery to a device. Samples may be treated to eliminate background cells or to solubilize viscous components of the sample. Samples may be pretreated to separate cells of interest from the source matrix or may be treated to stabilize cells of interest or enrich for cells of interest. For example, blood samples may be treated with an anticoagulant or may be treated to lyse blood cells selectively. Samples may also be filtered to remove non-cellular debris. Samples may also be diluted to decrease viscosity. Additional sample treatment procedures include permeabilization and fixation. Sample treatment may or may not occur on the device prior to analysis.

An exemplary lysis procedure for a blood sample involves contacting the sample with 9 parts 0.7% Tween-20, 0.01M sodium phosphate buffer, and proteinase from Aspergillus melleus (Amano/Sigma) and heating for 1 hour at 37° C. The solution may also be used at 1:1 with heating for 30 min at 37° C. Additional components of a lysis buffer may include lipase, cholesterol esterase, double stranded DNase, 0.1M sodium phosphate buffer, and different or additional detergents (e.g., saponin and Triton-X). It may be advantageous to perform the lysis in the device as illustrated in the CR-BSI example.

Internal Control

Methods of the invention may also employ an internal control cell or particle, e.g., that is added to the sample at the lysis stage. Examples of control organisms include Prototheca wickerhamii (a type of algae); Paracoccus yeeii (gram negative); and Bacillus sphaericus (gram positive rod). These methods would employ a probe for the control organism in each lane; the control organism may also react with other probes present (e.g., BacUni or G+ if the organism is a gram positive bacterium). The morphology of the control organism could serve as an added identifier. Use of an internal control allows for determination if the method is working properly and can be used to account for uneven sample splitting in a device.

### Example 1

### Yeast Analysis

A Candida Speciation Panel was designed to analyze the five most prevalent Candida species in blood-stream infections: C. albicans, C. glabrata, C. krusei, C. parapsilosis, and C. tropicalis. It also contained a universal yeast probe, which was used to determine if the sample was yeast. The Candida Speciation Panel uses the methanol based PNA FISH assay described in WO 2005/121373. The whole assay was automated and run in a device of the invention. The device contained six channels with six different yeast probes. The sample was loaded into the device, where the hybridization and wash took place. There was continuous flow during the hybridization and wash steps, which allowed the sample to flow towards a membrane. Once the assay was done, the membrane was viewed for positive yeast cells. The procedure is as follows.
1. Inoculate yeast species (from a fresh YM plate) into YM broth and grow approximately 4-6 hrs.
2. After 4-6 hrs. take 100 μL of broth culture and dilute into 1 mL of the methanol-hybridization buffer and load into the device of FIG. 1 at 600 μL/min.
3. Run the probe/hybridization solution through the device at a flow rate of 300 μL/min for 5 min. at 55° C. and then slow the flow rate to 20 μL/min. for 25 min. at 55° C.
4. Run the wash solution through the device at a flow rate of 300 μL/min. for 20 min. at 55° C.
5. Allow device to cool and then view fluorescence with a FITC or dual-band filter using a 20× objective.

### Reagents

The hybridization buffer includes methanol (50%), 0.1M sodium chloride, 0.025M Tris-HCL (pH 9.0), 0.1% sodium dodecyl sulfate (SDS), 0.5% Yeast Extract Solution, and DEPC water (to 100%). The wash buffer includes 0.025M sodium chloride, 0.005M Tris-HCl (pH 9.0), 0.1% Triton X-100, 0.05% (v/v) ProClin 300, and DEPC water (to 100%). The probes employed are provided in Table 3.

TABLE 3

| | | | |
|---|---|---|---|
| C. albicans | Can26S03 | Flu-OO-AGAGAGCAGCATGCA | SEQ ID No: 1 |
| C. glabrata | Cgla26S07k | Flu-OO-ACAGTCCCAAAGTGGT | SEQ ID No: 2 |
| C. krusei | Ckru26S02a | Flu-OO-CCTTCCACACAGACTC | SEQ ID No: 3 |
| C. parapsilosis | Cpar26S04d | Flu-OO-TAGGTCTGGGACATC | SEQ ID No: 4 |
| C. tropicalis | Ctro26S07f | Flu-OO-CCAACGCAATTCTCCT | SEQ ID No: 5 |

In the table, the sequences of the PNA probes are shown. Flu stands for fluorescein attached at the N-terminus of the PNA molecule, O stands for O-linker, a glycol linker of nine atoms (i.e., —NH(CH₂CH₂O)₂CH₂C(O)—) used to distance the fluorophore from the hybridization portion of the probe, and A, C, T, and G stand for PNA monomers carrying the corresponding base.

If the channel were positive, yeast cells with green fluorescence were present on the membrane in that channel.

The Candida Speciation Panel was screened against 10 reference strains representing 10 fungal species (Table 4).

TABLE 4

| | | Pan | | | | | |
|---|---|---|---|---|---|---|---|
| | | Fungal | C. albicans | C. glabrata | C. krusei | C. parapsilosis | C. tropicalis |
| Species | Strain | PNA | PNA | PNA | PNA | PNA | PNA |
| C. albicans | Y-17968 | POS | POS | NEG | NEG | NEG | NEG |
| C. glabrata | ATCC | POS | NEG | POS | NEG | NEG | NEG |
| | 15126 | | | | | | |
| C. krusei | NRRL | POS | NEG | NEG | POS | NEG | NEG |
| | Y-7550 | | | | | | |
| C. parapsilosis | ATCC- | POS | NEG | NEG | NEG | POS | NEG |
| | 22019 | | | | | | |
| C. tropicalis | ATCC- | POS | NEG | NEG | NEG | NEG | POS |
| | 750 | | | | | | |
| C. dublienisis | NRRL | POS | NEG | NEG | NEG | NEG | NEG |
| | Y-27201 | | | | | | |
| C. guilliermondii | NRRL | POS | NEG | NEG | NEG | NEG | NEG |
| | Y-324 | | | | | | |
| Candida kefyr | ATCC- | POS | NEG | NEG | NEG | NEG | NEG |
| | 4135 | | | | | | |
| C. lusitaniae | NRRL | POS | NEG | NEG | NEG | NEG | NEG |
| | Y-11827 | | | | | | |
| S. cerevisiae | ATCC- | POS | NEG | NEG | NEG | NEG | NEG |
| | 9763 | | | | | | |

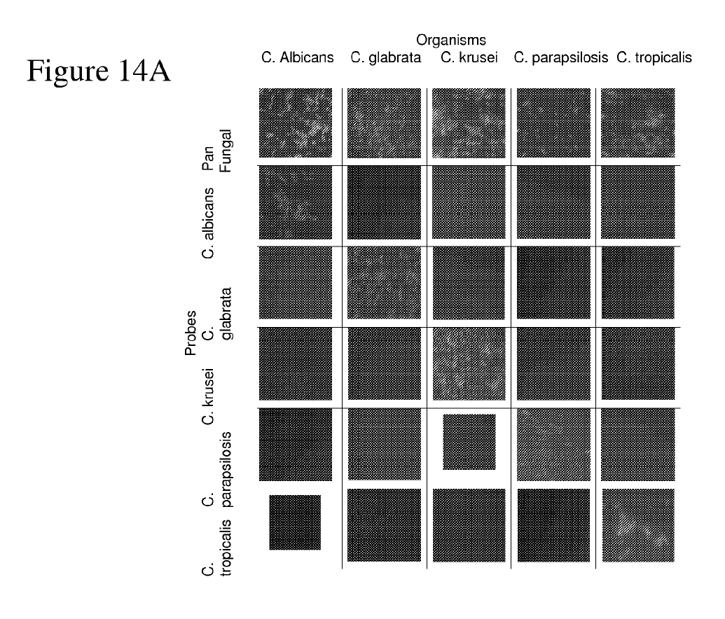

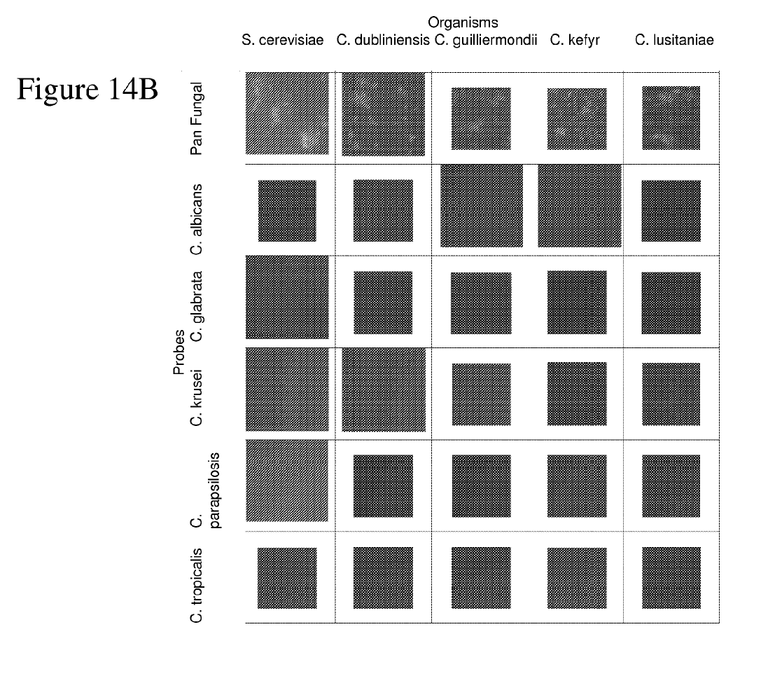

Results of the method for identifying yeasts are shown in FIGS. 14A-B.

An alternative operation of the assay in the system is as follows. The operator loads the sample into the device. The sample enters the device and flows through a splitter that sends equal aliquots of sample into each of the 6 reaction lanes. Once inserted into the system, a series of operations are performed on the device to process the sample. A blister containing degassed hybridization buffer is actuated. The actuation opens a burst valve and controlled flow of hybridization buffer begins. The flow drives the sample to the capture membranes, reconstitutes dried PNA, and moves the reagents over the captured cells on each membrane. The cells are hybridized under flow for 30 minutes at 55° C. Next, a blister containing degassed wash buffer is actuated. The actuation opens a burst valve and begins the flow. Wash buffer flows over the captured cells for 5 minutes at 55° C. Then device is cooled to less than 30° C. The membranes are scanned optically with an autofocus system that finds best focus for each field of view. The images are analyzed and scored based on cell fluorescence and cell morphology. The scoring is interpreted, and the test result is displayed. Images are stored for recall and review.

The method is intended for use in clinical microbiology laboratories to speciate yeast in isolates (liquid culture or colonies).

### Example 2

### CR-BSS

The CR-BSI test analyzes a sample for a panel of the most prevalent organisms responsible for catheter-related blood-stream-infections: S. aureus/CNS; E. faecalis/other Enterococci; EK (E. coli+K. pneumonia)/P. aeruginosa; C. albicans/other Candida. It also incorporates universal yeast and bacterial probes, which allow the analysis of organisms for which specific probes are not included. Gram+ and Gram− probes provide further information about bacteria detected with the universal probe. This method addresses an unmet clinical need for a point-of-care test for the diagnosis and management of catheter-related blood stream infections. The test may be performed in an Intensive Care Unit (ICU) and similar settings where catheterized patients are receiving care. The method analyzes, e.g., detects and speciates, bacteria and yeast in blood samples drawn from these patients (primarily through the catheter and potentially also peripheral draws).

The steps for this assay are summarized as follows (with reference to FIG. 5)
Load blood; cap; insert into analyzer
Actuate lysis solution; burst valve opens; lysis solution is delivered to blood chamber
Mix by driving blood/lysis solution back and forth to mix chamber (pneumatic drive via pressure ports); incubate
Drive lysed blood to membranes (pneumatic drive via pressure port)
Actuate push to complete delivery of sample to membranes and perform medium exchange; fix bacteria
Actuate hybridization buffer including analysis probes; deliver to membranes; incubate under slow flow
Actuate wash buffer; deliver to membranes; incubate under flow
Image

Ranges of specific temperatures, flow rates, and times are given in Table 5.

TABLE 5

| | | | Flow Rate | | |
|---|---|---|---|---|---|
| No. | Step | Fluid | or Vol | Temp | Time |
| 1 | Load sample | Blood | 1 mL | RT | |
| 2 | Mix lysis | Lysis | 1-9 mL | 37° C. | |
| | solution | solution | | | |
| 3 | Incubate | Lysis mix | 2-10 mL | 37° C. | 30-60′ |
| 4 | Drive to | Push | 600 μL/min | 37° C. | 3-16′ |
| | membrane | | | | |
| 5 | Heat fix | Push | | 80° C. | 2′ |
| | bacteria | | | | |
| 6 | Flow | HB, PNA, | 600 μL/min | 55° C. | <3′ |
| | hybridization | TCEP | | | |
| | buffer | | | | |
| 7 | Hybridization | HB, PNA, | 20 μL/min | 55° C. | 30′ |
| | | TCEP | | | |
| 8 | Wash | Wash buffer | 300 μL/min | 55° C. | ≦20′ |
| 9 | Image | | | <35° C. | <10′ |

HB = hybridization buffer;
Push is a non-methanol containing solution such as lysis reagent.
TCEP = tris(2-carboxyethyl)phosphine

A specific protocol is as follows:

| Number | Step | Temp | Time |
|---|---|---|---|
| 1 | Load blood, cap reservoir | RT | |
| 2 | Actuate lysis blister opening burst valve | 37° C. | |
| 3 | Mix by driving blood & lysis between blood | 37° C. | 1′ |
| | and mix chambers using pneumatic drive | | |
| | via vent ports | | |
| 4 | Incubate | 37° C. | 30′ |
| 5 | Drive lysed blood to membrane using | 37° C. | 1′ |
| | pneumatic drive | | |
| 6 | Open Vent 1, Close Vent 2 valve, | 37° C. | 10′ |
| | directing flow through membrane, | | |
| | complete delivery of lysed blood | | |
| 7 | Actuate Push blister, opening burs tvalve | 37° C. | |
| 8 | Flow Push, displacing plasma | 37° C. | 3′ |
| 9 | Fix bacteria | 80° C. | 2′ |
| 10 | Close pinch valves | | |
| 11 | Actuate hybe blisters, opening burst valves | 55° C. | |
| 12 | Flow of hybridization buffer through | 55° C. | 30′ |
| | hybridization step | | |
| 13 | Actuate wash blister, opening burst valve | 55° C. | |
| 14 | Flow wash buffer to complete wash step | 55° C. | 5′ |
| 15 | Image | <35° C. | |

Exemplary probes for a 4-channel CR-BSI assay are described schematically in FIGS. 11A-11F. Specific probes are shown in Table 6.

TABLE 6

| | | |
|---|---|---|
| S. aureus | Flu-OO-GCT-TCT- | SEQ ID No: 6 |
| | CGT-CCG-TTC | |
| CNS var. A | Tam-OO-AGA-CGT- | SEQ ID No: 7 |
| | GCA-TAG-T | |
| CNS var. B | Tam-OO-GCT-AAT- | SEQ ID No: 8 |
| | ACG-GCG | |
| E. faecalis | Flu-OO-CCT-CTG- | SEQ ID No: 9 |
| | ATG-GGT-AGG | |
| OE | Tam-OO-CCT-TCT- | SEQ ID No: 10 |
| | GAT-GGG-CAG | |
| E. Coli¹ | Flu-OO-TCA-ATG- | SEQ ID No: 11 |
| | AGC-AAA-GGT | |
| K pneumoniae¹ | Flu-OO-CAC-CTA- | SEQ ID No: 12 |
| | CAC-ACC-AGC | |
| P. aeruginosa | Tam-OO-CTG-AAT- | SEQ ID No: 13 |
| | CCA-GGA-GCA | |
| C. albicans | Flu-OO-AGA-GAG- | SEQ ID No: 1 |
| | CAG-CAT-GCA | |
| C. glabrata | Tam-OO-ACA-GTC- | SEQ ID No: 2 |
| | CCA-AAG-TGG-T | |
| C. krusei² | Tam-OO-CCT-TCC- | SEQ ID No: 3 |
| | ACA-CAG-ACT-C | |
| C. parapsilosis² | Tam-OO-TAG-GTC- | SEQ ID No: 4 |
| | TGG-GAC-ATC | |
| C. tropicalis² | Tam-OO-CCA-ACG- | SEQ ID No: 5 |
| | CAA-TTC-TCC-T | |
| BacUni | Cy5-OO-CTG-CCT- | SEQ ID No: 14 |
| | CCC-GTA-GGA | |
| PanFungal | Cy5-OO-CCC-TAG- | SEQ ID No: 14 |
| | TCG-GCA-TAG | |

¹EK;
²O Candida
Probes for Gram positive, Gram negative, and particular control organisms will be known to one skilled in the art.

### Other Embodiments

While the present invention has been described with reference to what are presently considered to be the preferred examples, it is to be understood that the invention is not limited to the disclosed examples. To the contrary, the invention is intended to cover various modifications and equivalent arrangements included within the spirit and scope of the appended claims.

All publications, patents and patent applications are herein incorporated by reference in their entirety to the same extent as if each individual publication, patent or patent application was specifically and individually indicated to be incorporated by reference in its entirety. Where a term in the present application is defined differently in a document incorporated herein by reference, the definition provided herein is to serve as the definition for the term.

Other embodiments are in the claims.

What is claimed is:

1. A device comprising a housing having a fluid inlet that is in fluid communication with a plurality of channels, wherein each of said plurality of channels is in fluid communication with a reservoir containing reagents for analyzing cells, particles, or analytes bound to said particles, one or more substantially planar, porous membranes through which said cells or particles do not pass, and one or more outlets, wherein liquid flowing away from said inlet is divided between said plurality of channels and flows through said one or more membranes towards said outlet, and wherein said reservoir is disposed upstream of said one or more membranes.

2. The device of claim 1, wherein said reagents for analyzing bind to a target analyte bound to said particles.

3. The device of claim 1, further comprising at least one reservoir for said particles, wherein said reservoir is disposed between said inlet and one of said one or more membranes, and wherein said reservoir for said particles is in fluid communication with at least one of said plurality of channels.

4. The device of claim 1, wherein said housing comprises a portion through which optical analysis of said cells or particles on one of said one or more membranes may occur.

5. The device of claim 1, further comprising electrodes disposed adjacent to or on one of said one or more membranes for electrical analysis of said cells or particles.

6. The device of claim 1, further comprising a magnetic resonance detector adjacent to one of said one or more membranes for magnetic relaxation analysis of said cells or particles.

7. The device of claim 1, further comprising a reservoir for waste disposed between one of said one or more membranes and said outlet.

8. The device of claim 1, further comprising a reservoir containing liquid reagents disposed between said inlet and one of said one or more membranes and separated from said plurality of channels by a valve.

9. The device of claim 1, further comprising a reservoir for containing a sample disposed between said inlet and one of said one or more membranes and in fluid communication with said plurality of channels.

10. The device of claim 9, further comprising a reservoir containing liquid reagents separated from said sample reservoir by a valve.

11. The device of claim 1, further comprising a temperature sensor and/or a heating or cooling element.

12. The device of claim 1, further comprising a passive mixer or an active mixer element disposed between said inlet and one of said one or more membranes.

13. The device of claim 1, wherein each of said plurality of channels has substantially the same fluidic resistance.

14. The device of claim 1, wherein, for at least one of said plurality of channels, said reservoir is disposed within said channel.

15. The device of claim 1, further comprising a plurality of reservoirs of reagents for analyzing, wherein each reservoir is in fluid communication with at least one of said plurality of channels.

16. The device of claim 15, wherein each of said plurality of reservoirs is in fluid communication with one, and only one, of said plurality of channels.

17. The device of claim 16, wherein each of said reservoirs is disposed within said channel.

18. The device of claim 1, wherein, for at least one of said plurality of channels, said reservoir is separated from said channel by a valve.

19. The device of claim 1, wherein said reservoir of reagents for analyzing is in fluid communication with each of said plurality of channels, so that flow of said reagents for analyzing away from said inlet is divided between said plurality of channels.

20. The device of claim 1, wherein one of said one or more membranes is substantially nonfluorescent.

21. The device of claim 1, wherein one of said one or more membranes is resistant to degradation by alcohol, acid, or base.

22. he device of claim 1, further comprising a plurality of reservoirs of reagents for analyzing, wherein each of said plurality of channels is separated from one of said reservoirs of reagents by a reservoir valve, and further comprising a plurality of channel valves that when closed prevent flow between said channel and said inlet.

23. The device of claim 22, further comprising a sample chamber and a liquid reagent chamber, wherein said sample chamber and said liquid reagent chamber are separated by a valve and are disposed between said inlet and said plurality of channels.

24. The device of claim 1, wherein said reservoir is a mechanically deformable chamber, and compression of said chamber expels its contents.

25. The device of claim 1, wherein said outlet allows the passage of gas but not liquid.

26. A system having a receptacle for mating to a device of any of the preceding claims and comprising (i) actuators for pumping fluids from said inlet of said device towards said outlet of said device; (ii) a temperature controller configured to interface with said device to control the temperature in at least a portion of said device; and (iii) a detector configured to interface with said device for analysis of cells, particles, or analytes bound to said particles on said membrane.

27. The system of claim 26, further comprising an active mixer element configured to interface with said device to mix two fluids between said inlet and said membrane.

28. The system of claim 26, wherein said detector is an optical detector, electrical detector, or a magnetic relaxation or magnetic resonance detector.

29. The system of claim 26, further comprising a reservoir for fluids and a pump to deliver fluids from said reservoir to said inlet of said device.

30. A method of analyzing a sample, said method comprising the steps of:
(i) introducing said sample into a device comprising a housing having a fluid inlet that is in fluid communication with a plurality of channels, wherein each of said plurality of channels is in fluid communication with a reservoir containing reagents for analyzing cells, particles, or analytes bound to said particles, one or more substantially planar, porous membranes through which said cells or particles do not pass, and one or more outlets, wherein liquid flowing away from said inlet is divided between said plurality of channels and flows through said one or more membranes towards said outlet, and wherein said reservoir is disposed upstream of said one or more membranes,
wherein said sample comprises said cells, particles or analytes that bind to said particles;
(ii) allowing said reagents for analyzing to contact said cells, particles, or analytes;
(iii) capturing said cells or particles on said membrane; and
(iv) analyzing said cells or particles on said membrane.

31. The method of claim 30, wherein said reagents for analyzing comprise probes for nucleic acids or antibodies.

32. The method of claim 31, wherein said probes comprise PNA, DNA, or LNA.

33. The method of claim 32, wherein said cells, particles, or analytes bound to said particles are further contacted with reagents that bind to said probes or antibodies, resulting in signal amplification.

34. The method of claim 30, wherein said reagents for analyzing are labeled for optical, electrical, or magnetic detection.

35. The method of claim 30, wherein said reagents for analyzing comprise a plurality of reagents that are optically distinguishable and that bind to different cells, particles, or analytes bound to said particles.

36. The method of claim 30, further comprising, after step (i) and prior to step (iv), treating said sample with a liquid reagent.

37. The method of claim 36, wherein said device further comprises a passive mixer disposed so that said liquid reagent and said sample mix while flowing through said device and before contacting said membrane.

38. The method of claim 36, wherein said treating step further comprises actively mixing said sample with said liquid reagent.

39. The method of claim 38, wherein said device further comprises a sample reservoir and a liquid reagent reservoir separated by a valve from said sample reservoir, wherein said sample is introduced into said sample reservoir in step (i), said liquid reagent is stored in said liquid reagent reservoir, and said active mixing comprises actuating said valve and transferring said liquid reagent to said sample reservoir or said sample to said liquid reagent reservoir.

40. The method of claim 36, wherein said liquid reagent comprises a diluent, lysis buffer, or said particles comprising binding moieties to said analytes.

41. The method of claim 40, wherein said sample is contacted with said particles comprising binding moieties under conditions in which said analytes of said sample bind to said particles, which are captured by said membrane in step (iii).

42. The method of claim 30, wherein said sample is contacted with control particles that are subsequently divided between said plurality of channels proportionally with said sample, wherein said control particles are captured by said membrane in step (iii).

43. The method of claim 30, wherein said device further comprises a plurality of reservoirs containing reagents for analyzing, wherein one of said plurality of reservoirs is disposed within each of said plurality of channels, and said reagents for analyzing are released from said reservoir by flow of adjacent liquid in step (ii).

44. The method of claim 30, wherein said device further comprises a plurality of reservoirs containing reagents for analyzing, wherein one of said plurality of reservoirs is separated by a valve from each of said plurality of channels, and step (ii) comprises actuating said valve.

45. The method of claim 30, wherein said cells are microorganisms and are analyzed.

46. The method of claim 30, wherein said cells are analyzed and are produced by a subject as a result of disease.

47. The method of claim 30, wherein said sample comprises a culture, an environmental sample, or a biological sample.

48. A kit comprising a device of claim 1 and a diluent, lysis buffer, hybridization buffer, or control particles.

49. A device that can detect, identify, and quantify a low-level of microorganisms in complex biological samples.


SEQUENCE LISTING (15 sequences)

SEQ ID NO: 1 - DNA, 15 bp, Artificial Sequence
  Synthetic Construct
agagagcagcatgca

SEQ ID NO: 2 - DNA, 16 bp, Artificial Sequence
  Synthetic Construct
acagtcccaaagtggt

SEQ ID NO: 3 - DNA, 16 bp, Artificial Sequence
  Synthetic Construct
ccttccacacagactc

SEQ ID NO: 4 - DNA, 15 bp, Artificial Sequence
  Synthetic Construct
taggtctgggacatc

SEQ ID NO: 5 - DNA, 16 bp, Artificial Sequence
  Synthetic Construct
ccaacgcaattctcct

SEQ ID NO: 6 - DNA, 15 bp, Artificial Sequence
  Synthetic Construct
gcttctcgtccgttc

SEQ ID NO: 7 - DNA, 13 bp, Artificial Sequence
  Synthetic Construct
agacgtgcatagt

SEQ ID NO: 8 - DNA, 12 bp, Artificial Sequence
  Synthetic Construct
gctaatacggcg

SEQ ID NO: 9 - DNA, 15 bp, Artificial Sequence
  Synthetic Construct
cctctgatgggtagg

SEQ ID NO: 10 - DNA, 15 bp, Artificial Sequence
  Synthetic Construct
ccttctgatgggcag

SEQ ID NO: 11 - DNA, 15 bp, Artificial Sequence
  Synthetic Construct
tcaatgagcaaaggt

SEQ ID NO: 12 - DNA, 15 bp, Artificial Sequence
  Synthetic Construct
cacctacacaccagc

SEQ ID NO: 13 - DNA, 15 bp, Artificial Sequence
  Synthetic Construct
ctgaatccaggagca

SEQ ID NO: 14 - DNA, 15 bp, Artificial Sequence
  Synthetic Construct
ctgcctcccgtagga

SEQ ID NO: 15 - DNA, 15 bp, Artificial Sequence
  Synthetic Construct
ccctagtcggcatag